(19) United States
(12) Patent Application Publication
(10) Pub. No.: US 2008/0039671 A1
(43) Pub. Date: Feb. 14, 2008

(54) Process for chemical conversions in membrane reactors and recovery of purified product

(75) Inventors: Michael J. Foral, Aurora, IL (US); Martin E. Carrera, Naperville, IL (US); Craig W. Colling, Warrenville, IL (US); Brian C. Bahr, Friendswood, TX (US); C. Francisco Lorenzano-Porras, Naperville, IL (US); Bruce D. Alexander, Lombard, IL (US)

Correspondence Address: INEOS USA LLC, 3030 WARRENVILLE RD, S/650, LISLE, IL, 60532, US

(73) Assignee: Innovene USA, Lisle, IL (US)

(21) Appl. No.: 11/408,303

(22) Filed: Aug. 11, 2006

(51) Int. Cl. C07C 5/327

(52) U.S. Cl. 585/654

(57) ABSTRACT

Processes for chemical conversion of volatile organic compounds to value added products using membrane reactors and recovery of one or more purified conversion product are described. Useful membranes are preselected to control the relative amount of noncondensable co-product in gaseous reactor effluent such that the energy required for the subsequent compression and partial condensation of the reactor effluent is reduced.

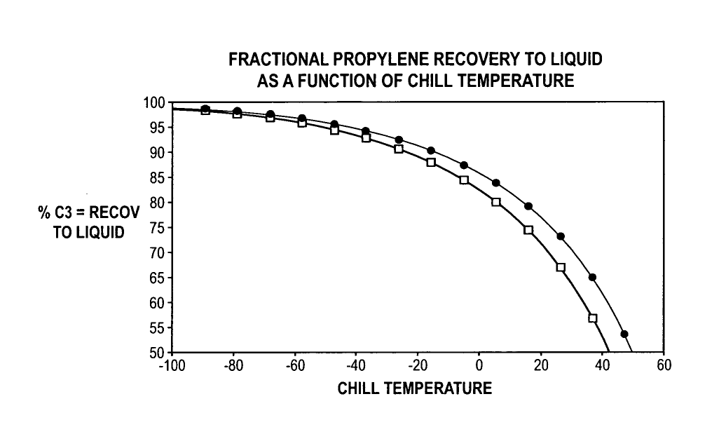

### TECHNICAL FIELD

The present invention relates to processes for chemical conversion of volatile organic compounds to value added products using membrane reactors and recovery of one or more purified conversion product. In particular, conversion processes of the invention provide a membrane reactor effluent stream that contains a gaseous mixture of value added organic products and non-condensable co-products such as dihydrogen. At least a portion of the gaseous effluent from the membrane reactor is compressed and cooled to form a liquid fraction rich in organic products and a noncondensable-rich gaseous fraction. Value added hydrocarbon products are recovered from the organic-rich liquid fraction. Useful membranes are preselected to control the relative amount of noncondensable co-product in the effluent stream such that the energy required for the subsequent compression and partial condensation of the reactor effluent is reduced.

### BACKGROUND OF THE INVENTION

Alkenes, commonly known as olefins, are used to produce many useful polymers and as components of numerous synthetic chemicals. Ethylene is used in one of several forms of polyethylene, as ethylene glycol to make polyester, in the manufacture of vinyl acetate and vinyl chloride, as a building block for linear alpha olefins, and in the production of styrene. Propylene is used in the synthesis of polypropylene, propylene oxide, acrylonitrile, and cumene. Butadiene is used primarily to make elastomers including styrene-butadiene rubber, neoprene, and nitrile rubber. The olefins/polymers value chain is typically composed of several distinct steps: (1) conversion of hydrocarbons including alkanes into alkenes, (2) in some cases transformation of the alkenes into intermediate products via oxidation, ammoxidation, or alkylation (e.g. acrylonitrile, styrene, and cumene), (3) polymerization or oligomerization into macromolecules, and (4) final device fabrication into end products.

Several commercialized methods are practiced to synthesize olefins. The most industrially significant method is steam cracking. Steam crackers can produce olefins from numerous hydrocarbon feeds including natural gas liquids, light petroleum gases, light paraffinic naphthas, and mixtures thereof. Commercialized steam cracking processes utilize high temperature pyrolysis where these feeds are mixed with steam and heated to temperatures in a range from about 700° to 900° C. Thermodynamic equilibrium limits olefin yield to relatively low amounts. The olefins industry has gotten above this constraint by pushing temperatures up to where free radical mechanisms start to occur. The industry relies on high temperatures and quick contact times so that free radical reactions are quickly quenched to focus the yield pattern on olefins and limit the formation of byproducts. Reactor development in conventional olefins crackers has been oriented toward shorter and shorter contact times with large quench heat exchangers to quickly stop the reactions. More detail regarding the operation, engineering, and optimization of steam cracking may be found in Ullmann's Encyclopedia of Industrial Chemistry.

When an olefin is made from an alkane, commonly known as paraffin, hydrogen is also produced. Thermodynamics dictates the maximum yield of olefins and hydrogen possible at a specific reactor temperature. Chemical conversions approach but do not exceed the thermodynamic equilibrium limit. See for example, U.S. Pat. No. 6,271,431, in the name of Christian Busson, Jean-Pierre Burzynski, Henri Delhomme, and Luc Nougier, discribes a reactor that produces ethylene yields higher than those normally obtained in commercial cracking reactors by lowering the temperature and increasing the contact time of the process. Their process approaches but cannot exceed the thermodynamic equilibrium limit.

U.S. Pat. No. 6,111,156, in the name of Michael C. Oballa, David Purvis, Andrzej Z. Krzywicki, and Leslie W. Benum, describes a high temperature, high conversion olefin process that approaches the maximum thermodynamic yield of olefins. The patent describes furnace tubes or coils that have been adapted to operate at temperatures higher than those typically employed in conventional steam crackers (above 1050° C.), thereby increasing conversion. Examples of these adaptations include coatings available from Surface Engineered Products and ceramic tubes including silicon carbide.

There are several problems with this approach to increasing olefin yield. Joining silicon carbide to metals is very difficult and the technology for doing so, and keeping the joint in tact at these temperatures (above 1050° C.), is not well developed. Therefore, this leads to frequent ceramic tube failures and generally unreliable operations. Furthermore, vibrations typically encountered during steam cracking operations can easily damage and destroy silicon carbide tubes at the elevated temperatures described in U.S. Pat. No. 6,111,156. Olefin selectivity is believed to be poor at these elevated temperatures. If the operation of the steam-cracking reactor is too severe, numerous researchers have pointed out that the amount of olefin produced per pound of feed converted can actually level out and even decrease. For example, a kinetic severity factor (KSF) is defined in “Pyrolysis: Theory and Industrial Practice” edited by L. F. Albright and coworkers and published by Academic Press in 1983 that relates reactor residence time, reactor temperature, reactor pressure, quenching, and feedstock type. They show that the concentration of olefins passes through a maximum as KSF is increased. This occurs because secondary reactions that begin to consume olefins play a larger role at high severity. The amount of undesirable byproducts is also understood to be high when steam cracking at the elevated temperatures described in U.S. Pat. No. 6,111,156.

Alternative routes for the production of ethylene, propylene and butylenes have been of interest for many years as an alternative to steam cracking. Note that all of these can approach but not exceed the thermodynamic conversion limit. The most feasible route to the commercial scale on-purpose production of these alkenes has generally been through the catalytic dehydrogenation of the relevant alkane according to the formula

CₙH₍₂ₙ₊₂₎——————————>CₙH₍₂ₙ₎+H₂

where n is an integer greater than or equal to 2. Catalytic dehydrogenation reactions are limited by thermodynamic constraints resulting from the highly endothermic nature of the reaction. As reactor temperatures increase above 600° C., side-cracking reactions based on free radical mechanisms can occur, leading to the formation of lighter hydrocarbons and coke. Employing a catalyst reduces the required reaction temperature and thereby largely avoids the formation of free radical species in the reactor. The high costs of the alkane feedstock (e.g., ethane, propane, etc.) and the capital required for the dehydrogenation processes make it economically desirable to achieve the highest possible selectivity to alkanes and to limit the formation of coke and coke precursors within the dehydrogenation reactor.

As in catalytic reforming, coke formation on and the resulting deactivation of the dehydrogenation catalyst is reduced by the addition of small amounts of dihydrogen to the dehydrogenation reactor feed. Significant research has been devoted to minimizing the coke formation reaction and in studying the kinetics of coke formation. For instance, R. L. Mieville (Studies in Surface Science and Catalysis, vol. 68, Catalyst Deactivation 1991, pp. 151-159) has showed that the rate of coke formation for a Pt/Al₂O₃ catalyst used in reforming obeys the following equation

rcoke=(A)*(1/pH2)*(pfeed^{0.75})*(1/coke)*(exp(−37000/RT))

Where pH₂ is the partial pressure of hydrogen, pfeed is the partial pressure of the hydrocarbon feed, and “coke” relates to the amount of coke already present on the catalyst. This equation shows that the rate of coke formation is inversely proportional to the hydrogen partial pressure. Without the addition of hydrogen, most dehydrogenation catalysts deactivate in a time frame that is not commercially viable. Typically, in catalytic dehydrogenation processes, the amount of hydrogen added with the reactant alkane for coke suppression is balanced against the reduction in equilibrium conversion brought about by the resulting higher hydrogen concentration. Even with hydrogen addition to the reactor feed, some coke is formed on the catalyst and all commercial catalytic dehydrogenation technologies employ a reactor configuration that is designed to include periodic regeneration of the catalyst.

The extent of the conversion of hydrocarbons to olefins in conventional dehydrogenation systems is typically limited by thermodynamic equilibrium. There is a need for processes that overcome this thermodynamic limit. Removal of this thermodynamic limitation would allow higher per-pass conversion of the hydrocarbon to take place, resulting in a more efficient overall process.

One method that can be employed to remove the thermodynamic limitation is to employ oxidative dehydrogenation of the alkane. Oxidative dehydrogenation of ethane to ethylene has been reviewed recently by Dai et al. (Current Topics in Catalysis, 3, 33-80 (2002)). In an oxidative dehydrogenation process oxygen is added to the dehydrogenation reactor feed and reacts with the hydrogen produced during the dehydrogenation reaction. The hydrogen is converted to water, thereby removing it from the reaction zone and driving the thermodynamic equilibrium to higher alkane conversion values. The heat provided by the exothermic oxidation of hydrogen also can balance the heat required by the endothermic dehydrogenation reaction.

While the concept of oxidative dehydrogenation is not new, to date the process has not been commercialized for the large-scale production of light olefins. There are a number of drawbacks to the use of oxidative dehydrogenation as compared to standard catalytic dehydrogenation. First, addition of oxygen to the feed typically leads to a reduced selectivity to the desired olefin product. Formation of carbon oxides and oxygenated compounds through the undesirable partial combustion of the hydrocarbon feed can lead to lower feed utilization and more complex downstream separation requirements for oxidative dehydrogenation processes. Second, mixing of oxygen with the hydrocarbon feed presents a safety concern that is not present in conventional catalytic dehydrogenation processes. While these risks can be mitigated through the application of safe engineering and design principles and additional safety systems, these systems and procedures can increase the cost and complexity of the oxidative dehydrogenation process and in any case the risks cannot be entirely removed. Finally, presence of both exothermic oxidation and endothermic dehydrogenation reactions within the reactor presents a significant reactor design challenge with regard to the management of heat within the reactor.

It is believed that the most promising way at present to remove the thermodynamic limitation of alkene production is to employ membranes capable of removing hydrogen. Removal of hydrogen causes the chemical reaction to proceed to the right through the law of mass action, thereby achieving much higher conversions, up to 100 percent conversion.

Membranes have been explored that remove hydrogen and thereby allow higher yields of olefins to be achieved. For example, U.S. Pat. No. 3,290,406 describes the use of palladium alloy tubes to remove hydrogen formed during the dehydrogenation of ethane. Membranes made out of palladium or palladium alloys are the most widely explored membranes for hydrogen separations. There are numerous reports in the art of palladium or palladium alloy membranes demonstrating high hydrogen permeation rates and hydrogen selectivities.

However, issues remain to be solved before palladium or palladium alloy membranes can be used in an industrial setting, as pointed out in an article by Collins and coworkers entitled “Catalytic Dehydrogenation of Propane in Hydrogen Permselective Reactors” in Industrial Engineering and Chemistry Research, volume 35, pages 4398-4405 (1996). Collins and coworkers found that palladium membranes deactivated rapidly when placed in alkane dehydrogenation service. Their membranes failed because of a large deposition of coke on the surface of the palladium membrane.

U.S. Pat. No. 5,202,517, in the name of Ronald G. Minet, Theodore T. Tsotsis and Althea M. Champagnie, appears to describe a way to overcome the coking problems associated with palladium membranes by use of porous ceramic membranes impregnated on the surface with palladium or platinum which are contacted with a mixture of alkane and hydrogen. They state that the hydrogen in the feed is needed to suppress the formation of coke.

Another way to suppress the formation of coke on the surface of a hydrogen membrane reactor is to supply a source of oxygen to the membrane reactor in the form of pure dioxygen (diatomic oxygen), air, or steam. However, supplying diatomic oxygen or air to the feed side of a hydrogen membrane reactor would suffer from the same drawbacks associated with oxidative dehydrogenation, namely safety concerns and reduced selectivity to the desired olefin product through the formation of carbon oxides.

It is therefore a general object of the present invention to provide an improved process which overcomes the aforesaid problem of prior art methods for chemical conversion of volatile organic compounds to value added products using membrane reactors.

An improved method for conversion of alkanes to corresponding alkenes should provide better ways to introduce oxygen into the reactor in order to keep the membrane free of coke.

There is a need for membranes that are capable of simultaneously transporting hydrogen and oxygen. For the synthesis of alkenes from alkanes, it is important to carefully balance the rates of oxygen and hydrogen transport. High hydrogen transport is desirable to maximize the production of olefins. Oxygen transport needs to be sufficient to prevent coking problems but not so high as to oxidize the alkane feedstock and form carbon oxides.

Membrane compositions have been described for transport of electrons and hydrogen. Other membrane compositions have been described for conducting electrons and oxygen. Membranes composed of a single phase capable of simultaneous hydrogen and oxygen transport have been described. For example, membranes composed of a single mixed oxide for oxygen and hydrogen transport are described in an article entitled “Oxide Ion Conduction in Ytterbium-Doped Strontium Cerate” by N. Bonanos, B. Ellis and M. N. Mahmood in Solid State Ionics, vol. 28-30, pages 579-579 (1988). A single phase mixed membrane for alkane dehydrogenation is described in U.S. Pat. No. 5,821,185, U.S. Pat. No. 6,037,514 and U.S. Pat. No. 6,281,403 each in the name of in the name of James H. White, Michael Schwartz and Anthony F. Sammels and assigned to Eltron Research, Inc.

U.S. Pat. No. 6,332,964 in the name of Chieh Cheng Chen, Ravi Prasad, Terry J. Mazanec and Charles J. Besecker describes membranes composed of an electron conducting phase and an oxygen-conducting phase. There are distinct advantages associated with employing such a matrix as a membrane in a reactor. A known problem in a reactor of this type is the slow buildup of coke on the alkane side of the reactor. Using a membrane matrix that conducts oxygen ions may reduce, or even eliminate, coking problems. It is believed that oxygen can be transported from the air side of the membrane to the alkane side where it may reacts with coke precursors as they are formed on the membrane surface. Reaction of the coke precursors with oxygen also provides heat to fuel the endothermic dehydrogenation reaction. Another use for the oxygen that is transported through such a matrix is to react with hydrogen to produce heat, as is needed in steam reforming.

U.S. Pat. No. 6,066,307 in the name of Nitin Ramesh Keskar, Ravi Prasad and Christian Friedrich Gottzmann describes a process for preparing synthesis gas and hydrogen gas using a membrane reactor having two membranes, one membrane that is an oxygen conductor and the other membrane that is a proton conductor, to produce hydrogen gas and synthesis gas.

Dual phase membranes offer the potential to balance the rates of oxygen and hydrogen transport. If one phase is responsible for hydrogen transport and the other is responsible for oxygen transport, it would be possible to adjust the relative amounts of the two phases to maximize hydrogen transport while maintaining an oxygen transport rate sufficient to keep the membrane from coking but not so high as to oxidize the alkane feedstock. It is harder to achieve this balance in single-phase membranes. There is a need for dual phase membranes that conduct both hydrogen and oxygen in order to produce a membrane reactor that facilitates chemical conversions without deactivating too rapidly. For example, improvements in steam reforming and alkane dehydrogenation would be expected if these dual phase membranes were employed.

Alkene production technologies described above produce, along with the desired olefin products, a variety of unwanted or lower-value co-products. For example, in the cracking of hydrocarbons to produce ethylene and propylene, coproducts such as methane, hydrogen, acetylene, and others are produced. Likewise, dehydrogenation of alkanes to produce the corresponding olefin also produces coproducts such as hydrogen, diolefins and acetylenics. Such coproducts make necessary one or more separation and purification steps so that a purified olefin product, suitable for downstream processing, can be obtained.

There is extremely wide scope for the design of such separation systems, limited only by the purity specifications of the final olefins product, technical feasibility, and economic viability. In a practical sense, however, such separation systems have nearly all contained at least the steps of compression of the olefin-containing reactor effluent stream, chilling and partial condensation of the compressed stream, and vapor/liquid separation wherein the liquid contains the olefin product and the vapor contains less valuable lighter gases.

For example with respect to separation of products from the dehydrogenation of aklanes, U.S. Pat. No. 6,333,445 in the name of John V. O'Brien described a cryogenic separation system for the recovery of olefins from a dehydrogenation process. This process included compression of the dehydrogenation reactor effluent and multiple levels of chilling and subsequent vapor/liquid separation. U.S. Pat. No. 5,026,952 in the name of Heinz Bauer describes a process for recovering C₂+, C₃+ or C₄ hydrocarbons from a high-pressure stream containing these components and lighter components. This process included multiple chilling and vapor/liquid separation stages, as well as rectification and expansion steps. U.S. Pat. No. 5,177,293 in the name of Michael J. Mitariten and Norman H. Scott describes a process for the separation and recovery of product streams from a dehydrogenation reactor that uses a pressure swing adsorption process to concentrate the olefin product. This process also comprises the steps of compression of the dehydrogenation reactor effluent, chilling of the compressed effluent, and subsequent vapor/liquid separation.

With respect to the separation of products from a hydrocarbon cracking reactor, a variety of commercial processes are offered by various technology vendors, including ABB Lummus Global, Kellogg Brown & Root, Inc., Linde A. G., Stone and Webster, Inc., and Technip-Coflexip, among others. A summary of the commercially available steam cracking and product purification technologies from these vendors has been published recently (Hydrocarbon Processing, March 2003, pp 96-98). While there are many and significant differences in the ethylene production and recovery processes offered by these vendors, it is clear to those skilled in the art that each of the ethylene production and purification processes contains at least the steps of compression of the furnace effluent, and the subsequent chilling and partial condensation of the compressed effluent to produce at least one olefin-rich liquid stream.

It is clear from the discussion above that the majority of separation processes which are necessary in the production of a purified olefin product from a reactor effluent comprise the steps of a) compression of the majority of the reactor effluent, b) chilling and partial condensation of the majority of the compressed reactor effluent stream, and subsequent separation of the resulting liquid and vapor phases to produce an olefin-rich liquid. These steps are common to essentially all light olefin-producing processes primarily because in the cases of dehydrogenation and steam cracking the reactor effluent exits the reactor at relatively low pressure, and because the subsequent purification of the desired olefin product is carried out largely through distillation and vapor/liquid separation. It would therefore be highly desirable to provide a reactor effluent stream which allows the subsequent steps of compression, chilling with partial condensation, and vapor/liquid separation to be carried out in a more energy- and capital-efficient manner.

Commercialized steam cracking processes utilize high temperature pyrolysis where these feeds are mixed with steam and heated to 700° C. to 900° C. During the process, less than 100 percent of the feed is converted per pass because of thermodynamic limitations and in order to maximize the yield of the desired olefin product. Complex and expensive separation equipment is used to recover the unreacted feed from the olefin products and byproducts. The unconverted feed is recycled where it is often remixed with fresh feed. This recycle introduces inefficiency to the process. It would be desirable to reduce this inefficiency by increasing per-pass conversion and thereby reducing or even eliminating recycles. Benefits of higher conversion olefins processes include reducing the size of equipment and capital employed in olefins manufacture, lowering the energy required to produce the olefin product, and elimination of large pieces of equipment devoted to separating the olefin product from unreacted alkane.

Other objects and advantages of the invention will become apparent upon reading the following detailed description and appended claims.

### SUMMARY OF THE INVENTION

In broad aspect, the present invention includes processes for chemical conversion of volatile organic compounds to value added products using membrane reactors and recovery of one or more purified conversion product. Useful membranes are preselected to control the relative amount of noncondensable co-product in the effluent stream such that the energy required for the subsequent compression and partial condensation of the reactor effluent is reduced. Economical processes are disclosed for chemical conversion of volatile organic compounds to value added products using dense membranes of multiphasic materials that provide independent, controllable, counter-current transport of hydrogen, electrons and oxygen.

In one aspect the invention is a process for chemical conversion of volatile organic compounds to value added products, which process comprises: providing a flow reactor comprising plurality of reaction zones each having at least one inlet for flow of fluid in contact with a solid state membrane and at least one outlet for flow of effluent from the reaction zone; Introducing a feedstream comprising volatile alkane compounds, substantially free of dihydrogen and dioxygen into all or a portion of the reaction zones; converting, at elevated temperatures, one or more volatile compound in the feedstream to products of conversion comprising corresponding value added organic products, carbonaceous co-products, and hydrogen; permitting at least a portion of the hydrogen co-product to be selectively conveyed out of one or more of the reaction zones through the solid membrane, thereby obtaining a gaseous effluent from the reaction zone that is characterized by a Relative Hydrogen Index value of less than 1.0; and compressing at least a portion of the gaseous effluent from the reaction zone, cooling the compressed effluent gas to form a liquid fraction rich in organic products and a dihydrogen-rich gaseous fraction, and recovering value added hydrocarbon products from the organic-rich liquid fraction.

In processes of the invention, one class of useful membranes comprise at least one metal selected from the group consisting of silver, palladium, platinum, gold, rhodium, titanium, nickel, ruthenium, tungsten, and tantalum. In another class, the membranes comprise a ceramic selected from the group consisting of a praseodymium-indium oxide mixture, niobium-titanium oxide mixture, titanium oxide, nickel oxide, tungsten oxide, tantalum oxide, ceria, zirconia, magnesia, or a mixture thereof.

In a particular useful class, the membrane comprises a multiphasic composition which in the form of a solid state membrane demonstrates an ability to selectively convey electrons, hydrogen and oxygen between different gaseous mixtures, the multiphasic composition comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity. These multiphasic, solid state membranes, advantageously demonstrates an ability to simultaneously convey a flux of hydrogen and, counter-current thereto, a flux of oxygen.

In another aspect of the invention using these multiphasic membranes the process further comprises permitting a predetermined amount of oxygen, in the form of oxygen ions, to be selectively conveyed into the reaction zone through the solid state membrane. Beneficially the flux of oxygen conveyed through the membrane into the reaction zone through the solid state membrane is no more than about one-fifth, of the counter-current hydrogen flux.

In another aspect, the invention is a process for chemical conversion of volatile organic compounds to value added products, which process comprises: providing a flow reactor comprising one or more reaction zone having at least one inlet for flow of fluid and at least one outlet for flow of effluent from the reaction zones; introducing a feedstream comprising volatile alkane compounds, substantially free of dihydrogen and dioxygen into all or a portion of the reaction zones; converting, at elevated temperatures, one or more alkane hydrocarbon in the feedstream to corresponding value added alkene hydrocarbons, carbonaceous co-products, and hydrogen; and permitting at least a portion of the hydrogen co-product to be selectively conveyed from the reaction zone through a dense, hydrogen-permeable membrane, thereby obtaining an effluent from the reaction zone that is characterized by a Relative Hydrogen Index value of less than 1.0.

This reactor effluent composition range is quantified by the “Relative Hydrogen Index” of the effluent, which is defined as a ratio of a difference between the total flow of hydrogen atoms and the flow of hydrogen atoms in the form of water in the effluent stream to the same difference for the feedstream.

In one aspect this invention further comprises permitting a predetermined amount of oxygen, in the form of oxygen ions, to be selectively conveyed into the reaction zone through a dense, oxygen-permeable membrane. In another aspect processes of the invention further comprise compressing at least a gaseous portion of the effluent from the reaction zone, and cooling the compressed effluent gas to form an alkene-rich liquid fraction and a dihydrogen-rich gaseous fraction; and recovering value added alkene hydrocarbon products from the organic-rich liquid fraction.

In yet another aspect of processes of the invention further comprise compressing at least a gaseous portion of the reaction zone effluent to provide a compressed effluent gas at an absolute pressure greater than 1.5 times the absolute pressure of uncompressed effluent, and cooling the compressed effluent gas to form a organic-rich liquid fraction and a dihydrogen-rich gaseous fraction.

In one aspect, the invention is a process for chemical conversion of volatile organic compounds to value added products, which process comprises: providing a flow reactor comprising one or more reaction zone having at least one inlet for flow of fluid and at least one outlet for flow of effluent from the reaction zones; introducing a feedstream comprising volatile alkane compounds, substantially free of dihydrogen and dioxygen into all or a portion of the reaction zones; converting, under conditions of conversion including elevated temperatures, at least 75 percent of one or more alkane hydrocarbon in the feedstream to corresponding value added alkene hydrocarbons, carbonaceous co-products and hydrogen; permitting at least a portion of the hydrogen co-product to be selectively conveyed from the reaction zone through a dense, hydrogen-permeable membrane, thereby obtaining an effluent from the reaction zone that is characterized by a Relative Hydrogen Index value of less than 1.0; and compressing at least a gaseous portion of the effluent from the reaction zone, cooling the compressed effluent gas to form a liquid fraction rich in organic products and a dihydrogen-rich gaseous fraction, and recovering value added hydrocarbon products from the organic-rich liquid fraction.

Particularly useful feedstreams comprise one or more volatile alkane compound having from about 1 to about 8 carbon atoms and the conversions are carried out at elevated temperatures in a range from about 400° C. to about 900° C. and pressures in a range upward from about 15 psia to about 500 psia.

For hydrocarbon dehydrogenation and oligomerization reactions, suitable catalysts include; oxides of the first row transition metals on a support, for example on an alkali metal oxide; one or more metal selected from the group consisting of nickel, iron platinum, silver and palladium; and perovskite compounds represented by the formula

(D_{1-y}M_{y})EOδ

where D is a metal selected from the group consisting of cerium, terbium, praseodymium and thorium; M comprises at least one other metal selected from the group consisting of titanium, vanadium, chromium, manganese, iron, cobalt, copper and nickel; and E is at least one element selected from the group consisting of magnesium, calcium, strontium, and barium; y is a number such that

0.02<y<0.5

and δ is a number that renders the compound charge neutral.

The recovered value added hydrocarbon products beneficially comprise at least member of the group consisting of ethylene, propylene, and isomers of butene.

In yet another aspect of the invention the membranes comprise a multiphasic composition which in the form of a solid state membrane demonstrates, under conditions of conversion, an ability to selectively convey electrons, hydrogen and oxygen between different gaseous mixtures. These multiphasic compositions advantageously comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrate electronic conductivity.

The flux of the co-product hydrogen conveyed through the membrane, out of the reaction zone beneficially is at least 1 cm³/min. at standard conditions per cm² of membrane area. Generally according to the invention, the flux of oxygen conveyed through the membrane into the reaction zone is no more than the counter-current flux of hydrogen. Advantageously, the flux of oxygen conveyed through the membrane is no more than about one-fifth of the counter-current hydrogen flux, and even less than one-tenth for best results.

In processes according to the invention the feedstream advantageously comprises one or more volatile alkane compound having from about 1 to about 8 carbon atoms, and the conversions are carried out at elevated temperatures in a range from about 400° C. to about 900° C. and pressures in a range upward from about 15 psia to about 150 psia.

Beneficially, recovery of value added hydrocarbon products from the organic-rich liquid fraction provides a stream of unconverted alkane hydrocarbon and a stream of corresponding purified alkene hydrocarbon, and the process further comprises introducing all or a portion of the unconverted alkane stream into at least a portion of the reaction zones.

### BRIEF DESCRIPTION OF THE DRAWING

The appended claims set forth those novel features which characterize the present invention. The present invention itself, as well as advantages thereof, may best be understood, however, by reference to the following brief description of preferred embodiments taken in conjunction with the annexed drawing, in which:

The FIGURE depicts the fractional recovery of propylene to the liquid phase following partial condensation of high-RHI and low-RHI propane dehydrogenation reactor effluents.

### BRIEF DESCRIPTION OF THE INVENTION

This invention relates to improving the downstream processability of the effluent of an olefins-producing reactor. In all commercial olefin-producing technologies, including steam cracking and alkane dehydrogenation, a mixture of products is formed so that the desired olefin product must be recovered from the reactor effluent and purified from other components which exist in the reactor effluent. Essentially all of the commercial olefin recovery and purification systems in use today involve at least both the compression and partial condensation of the reactor effluent. This invention identifies a particular reactor effluent composition range within which the energy required for the subsequent compression and partial condensation of the reactor effluent is reduced. This reactor effluent composition range is quantified by the “Relative Hydrogen Index” of the effluent, which is defined as a ratio of a deference between the total flow of hydrogen atoms and the flow of hydrogen atoms in the form of water in the effluent stream to the same difference for the feedstream. The Relative Hydrogen Index is represented by the equation:

RHI=(H_{T}−H_{W})_{E}/(H_{T}−H_{W})_{F}

where RHI is the Relative Hydrogen Index, the E subscript refers to the flow reactor effluent before any cooling or other processing, the F subscript refers to the reactor feed, H_{T} is the total flow of hydrogen atoms in the stream, and H_{W} is the flow of hydrogen atoms in the form of water vapor in the stream. For process for chemical conversion of volatile organic compounds to value added products according to the invention, membrane reactor effluents have an RHI of less than 1.0, and beneficially less than 0.75, by which savings of a significant amounts of energy are obtained in subsequent product recovery steps.

Such low-RHI reactor effluents can be produced by membrane reactors that transport oxygen ions, hydrogen ions, or both oxygen and hydrogen ions. Examples are given which demonstrate that current technologies which convert alkanes to olefins, including steam cracking and dehydrogenation, produce effluents with an RHI equal to or greater than 1.0, while such membrane reactors can produce effluents with an RHI of less than 1.0.

As an example, consider a pure dehydrogenation reaction. With this reaction, for every mole of carbon-carbon double bonds produced in the reactor from alkanes, one mole of hydrogen is produced. In addition, for every mole of carbon-carbon triple bonds produced in the reactor from alkanes, two moles of hydrogen are produced. In this case the nature of the reaction stoichiometry requires that the RHI of the reactor effluent to be at or near 1.0. Slight departures from an RHI of 1.0 in particular instances could be attributable to, for example, the formation of small amounts of coke in the dehydrogenation reactor.

The Relative Hydrogen Index can be measured for any olefin-producing reactor. For example, the RHI of the effluent of a steam-cracking furnace, the most common type of reactor for the commercial production of light olefins, can be measured. We have found that, over a wide range of reactor conditions, conversion levels, and feedstocks the RHI for steam cracking is relatively constant and very near 1.0.

A reactor effluent with a significantly different RHI is produced by membrane-based olefin production reactors, in particular those which transport oxygen ions, hydrogen ions, or both oxygen and hydrogen ions. In the case of an oxygen-transport membrane, oxygen transported to the reaction zone is allowed to react preferentially with hydrogen within the reaction mix, converting it to water and thereby removing it as dihydrogen from the reactor effluent. In the case of a hydrogen-transport membrane, hydrogen is transported from the reaction mix to the other side of the membrane, thereby removing it from the reactor effluent. In both cases the removal of dihydrogen from the reactor effluent will reduce the RHI of the reactor effluent to a value below 1.0.

A similar hydrogen removal effect can be obtained if molecular oxygen is added to the reactor feed so that hydrogen produced in the production of alkenes is removed through reaction with oxygen to form water. Such a process is known as oxidative dehydrogenation. While oxidative dehydrogenation offers the same hydrogen removal benefits as the membrane reactors described above, the reaction of oxygen is not completely selective to hydrogen removal. As a result some of the molecular oxygen reacts with alkane feed or alkene products to form carbon oxides such as carbon monoxide or carbon dioxide. Production of such carbon oxides is detrimental in three ways. First, non-selective oxidation converts at least some of the feed to relatively low-value carbon oxide products. Second, production of carbon oxides complicates the olefin purification process in that extra steps, such as carbon dioxide removal, may be required. Finally, the presence of carbon monoxide in particular is detrimental to the chilling and partial condensation of the reactor effluent in a manner similar to hydrogen itself, the detrimental effect of which is discussed hereunder.

Moreover, the mixing of oxygen with the hydrocarbon feed and subsequent heating of the mixture entails significant safety concerns since oxygen and hydrocarbons can form flammable and explosive mixtures. This concern is relevant in normal plant operation, but particularly so during upset conditions where the flowrates of oxygen and hydrocarbon may be difficult to control. For these reasons it would be desirable to achieve the hydrogen removal effect provided by oxidative dehydrogenation without resorting to the addition of molecular oxygen to the reactor feed. This desirable hydrogen removal effect can be achieved without oxygen addition through the use of the membrane reactors described above.

The primary benefit of a reactor effluent with a lower RHI is that it requires less energy to recover a purified olefin product from it than a reactor effluent with a higher RHI. This benefit is manifested both in the compression of the reactor effluent, and in the chilling and partial condensation of the compressed reactor effluent.

Other things being equal, for a given molar flow of the desired olefin product, a reactor effluent with a lower RHI will have less hydrogen and therefore a higher molecular weight than a reactor effluent with a higher RHI. As is well known to those skilled in the art of hydrocarbon processing, a gas with a higher molecular weight has a higher compressibility than a gas with a lower molecular weight. Put another way, for a given molar flow of the desired olefin product, a reactor effluent with a lower RHI will require less compressor power than a reactor effluent with a higher RHI. In addition, if in addition to the olefin flow rate the reactor effluent pressure and temperature are also constant, a reactor effluent with lower RHI will have a lower volumetric flow rate than a reactor effluent with higher RHI. In such a case the compressor will be smaller for the low-RHI reactor effluent than the high-RHI reactor effluent, resulting in lower compressor capital cost for the low-RHI reactor effluent.

In addition to the benefits arising when the reactor effluent is compressed, a relatively lower RHI reactor effluent will save energy when the compressed reactor effluent is chilled and partially condensed. The presence of light gases, such as dihydrogen and oxides of carbon, decreases the temperature at which liquid can condense. It is believed that a presence of dihydrogen lowers the temperature at which a hydrocarbon vapor condenses in the same way that it lowers the temperature at which a hydrocarbon liquid vaporizes (See U.S. Pat. No. 5,711,919 and EP 840,079 where dihydrogen was added to a vessel in which liquid hydrocarbon was being vaporized in order to reduce the temperature of the hydrocarbon vaporization.)

From this is becomes apparent that a lower-RHI reactor effluent, with less hydrogen present, will condense at a higher temperature than a higher-RHI reactor effluent. Put another way, at a given reactor effluent pressure a lower-RHI reactor effluent will begin to condense at a higher temperature and will have a greater fraction condensed at any given temperature than a higher-RHI reactor effluent. It is well known to those skilled in the art of olefin recovery that relatively more energy is required to provide refrigeration at a lower temperature than at a higher temperature. Therefore an olefin-rich product can be obtained from a lower-RHI reactor effluent at a higher temperature and therefore with less energy than from a higher-RHI reactor effluent.

Those skilled in the art will recognize that there is the opportunity to optimize these two benefits of a lower-RHI reactor effluent gas. In particular, compressing the reactor effluent to a higher pressure will both increase the required compressor power and reduce the required refrigeration energy needed in the subsequent chilling step or steps. Alternately, the reactor effluent can be compressed to a lower pressure, consequently saving on compressor power while increasing the required refrigeration energy. The optimum combination of compression and refrigeration power will be determined by the cost of energy, the capital cost of the compressors, drivers, and refrigeration equipment, and other factors. It is a benefit of this invention that the processing of a lower-RHI reactor effluent will have lower total energy (defined as the sum of the reactor effluent compressor energy and the refrigeration energy) than the processing of a higher-RHI reactor effluent.

The following examples will serve to illustrate certain specific embodiments of the herein-disclosed invention. These examples should not, however, be construed as limiting the scope of the novel invention, as there are many variations which may be made thereon without departing from the spirit of the disclosed invention, as those of skill in the art will recognize.

### EXAMPLES OF THE INVENTION

### General

This invention is described by way of a number of relevant examples. Comparative examples show that conventional commercial olefins-producing technologies produce reactor effluent compositions with an RHI equal to or greater than 1.0. Examples of the invention demonstrate that suitable membrane-based reactors produce reactor effluent compositions having values of RHI less than 1.0. These Examples of the invention demonstrate that reactor effluents with lower RHI values advantageously require less energy to compress effluent and partially condense value added products than for the reactor effluents with higher RHI values.

### Comparative Example A

This comparative example demonstrates that the effluent from a conventional catalytic dehydrogenation reactor has an RHI value of at least 1.0, which values exceeded RHI values of this invention. Data was taken from U.S. Pat. No. 6,313,063 that demonstrated operation of conventional, catalytic, propane dehydrogenation at RHI values greater than 1.0. A magnesium hydrotalcite catalyst containing platinum (Pt), and tin (Sn) was evaluated under dehydrogenation conditions of 1 bar absolute pressure, 600° C., Gas Hourly Space Velocity of 2100 hr-1 with a feedstock consisting of 35 NmL/min propane, 5 NmL/min H2, 25 NmL/min N2, and 41 NmL/min steam (see Example 7). This resulted in a RHI of 1.0 for this example, and a propane-to-dihydrogen mol ratio of 7. The data given in the patent shows that this catalyst is stable over 25 hrs under these conditions resulting in 58 percent propane conversion at greater than 93 percent selectivity. While this example is typical for this particular catalytic dehydrogenation technology, propane-to-dihydrogen ratios of between 1 and 10 have been used in other catalytic, propane dehydrogenation technologies. In all cases however, the RHI was greater than or equal to 1.0, which values exceeded RHI values of this invention.

### Comparative Example B

This example demonstrates that the effluent from a conventional stream cracking furnace had an RHI of value of at least 1.0, and that this was true for several furnace feedstocks of commercial interest. In theory the RHI of a commercial steam cracking furnace effluent can be measured directly by sampling both the furnace feed and the furnace effluent. However, in practical terms is difficult both because the furnace effluent is very hot and difficult to handle, and because the furnace effluent composition is very complex and difficult to analyze accurately, particularly the heavier hydrocarbon components. The data for this example, therefore, is taken from the results of an accurate proprietary kinetic model of a commercial steam-cracking furnace. This model rigorously models the free radical reactions occurring in the cracking furnace and provides detailed furnace effluent compositions that are readily analyzed. This model has been validated against numerous commercial furnace tests and can therefore be considered a reliable predictor of actual furnace performance.

The model was used to simulate the performance and yields of steam cracking furnaces processing pure ethane, pure naphtha, and pure propane. The steam-to-hydrocarbon mass ratios were 0.28, 0.5, and 0.3 in the feeds to the ethane, naphtha, and propane furnaces, respectively. Furnace feed and effluent compositions are given in Table I, along with the calculated molar hydrogen flows in these streams. The molar atomic hydrogen flows were calculated for each component through the use of the formula

Mol_{H}=(Mass_{C}/M_{wtC})(Mol_{H}/Mol_{C})

Where the term Mol_{H} is the molar atomic flow of hydrogen, Mass_{C} is the mass flow of the component, the term M_{wtC} is the molecular weight of the component, and the ratio Mol_{H}/Mol_{C} is the number of hydrogen atoms in the component molecule. Over 100 distinct and lumped components were tracked by the model, and these are simplified to the categories shown in Table I.

Using the formula for RHI given above, the RHI for the effluents from the ethane, naphtha, and propane furnaces are 1.000, 1.003, and 1.001, respectively. These are all very near to 1.0, demonstrating that over a wide range of feedstocks, conventional steam cracking furnaces produce an effluent with an RHI of values of at least 1.0, which values exceeded RHI values of this invention.

TABLE I
Furnace Feed and Effluent Data for Example B

| | Furnace Feed | | | | | | Furnace Effluent | | | | | |
|---|---|---|---|---|---|---|---|---|---|---|---|---|
| | Mass Flow | | | Hydrogen Mole Flow | | | Mass Flow | | | Hydrogen Mole Flow | | |
| | (lb/hr) | | | (lbmol/hr) | | | (lb/hr) | | | (lbmol/hr) | | |
| Compo- | Ethane | Naphtha | Propane | Ethane | Naphtha | Propane | Ethane | Naphtha | Propane | Ethane | Naphtha | Propane |
| nent | Cracker | Cracker | Cracker | Cracker | Cracker | Cracker | Cracker | Cracker | Cracker | Cracker | Cracker | Cracker |
| H2O | 109,972 | 330,402 | 162,775 | 12,206 | 36,671 | 18,066 | 109,270 | 328,433 | 161,811 | 12,128 | 36,452 | 17,959 |
| CO | 0 | 0 | 0 | 0 | 0 | 0 | 783 | 2,870 | 1,345 | 0 | 0 | 0 |
| CO2 | 0 | 0 | 0 | 0 | 0 | 0 | 88 | 218 | 109 | 0 | 0 | 0 |
| Hydrogen | 0 | 0 | 0 | 0 | 0 | 0 | 16,945 | 7,487 | 9,405 | 16,777 | 7,413 | 9,312 |
| Methane | 0 | 0 | 0 | 0 | 0 | 0 | 19,012 | 92,039 | 119,038 | 4,741 | 22,952 | 29,685 |
| Acetylene | 0 | 0 | 0 | 0 | 0 | 0 | 1,605 | 6,939 | 4,311 | 123 | 533 | 331 |
| Ethylene | 0 | 0 | 0 | 0 | 0 | 0 | 201,370 | 201,370 | 201,370 | 28,716 | 28,716 | 28,716 |
| Ethane | 392,759 | 0 | 0 | 78,369 | 0 | 0 | 125,478 | 17,778 | 19,183 | 25,037 | 3,547 | 3,828 |
| MAPD | 0 | 0 | 0 | 0 | 0 | 0 | 103 | 6,622 | 2,427 | 10 | 661 | 242 |
| Propylene | 0 | 0 | 0 | 0 | 0 | 0 | 6,182 | 87,803 | 81,303 | 881 | 12,519 | 11,593 |
| Propane | 0 | 0 | 542,583 | 0 | 0 | 98,450 | 808 | 1,648 | 54,258 | 147 | 299 | 9,845 |
| Butadiene | 0 | 0 | 0 | 0 | 0 | 0 | 5,602 | 32,856 | 15,611 | 603 | 3,580 | 1,695 |
| Butanes | 0 | 0 | 0 | 0 | 0 | 0 | 757 | 89 | 94 | 130 | 15 | 16 |
| Butenes | 0 | 0 | 0 | 0 | 0 | 0 | 791 | 18,055 | 5,662 | 113 | 2,575 | 807 |
| Pentanes | 0 | 183,783 | 0 | 0 | 30,567 | 0 | 0 | 1,933 | 0 | 0 | 322 | 0 |
| Pentenes | 0 | 11,220 | 0 | 0 | 1,600 | 0 | 3,089 | 17,356 | 7,814 | 312 | 1,885 | 816 |
| C6 | 0 | 190,523 | 0 | 0 | 27,822 | 0 | 4,897 | 68,172 | 11,612 | 384 | 5,387 | 927 |
| Com- | | | | | | | | | | | | |
| pounds | | | | | | | | | | | | |
| C7+ | 0 | 275,278 | 0 | 0 | 37,711 | 0 | 5,952 | 99,537 | 10,004 | 401 | 7,630 | 736 |
| Total | 502,731 | 991,206 | 705,358 | 90,574 | 134,370 | 116,516 | 502,731 | 991,206 | 705,358 | 90,504 | 134,487 | 116,510 |

### Example 1

This example demonstrates that effluent from membrane-based reactors of the invention have values of RHI that are significantly less than 1.0.

A multiphasic, solid state electron, hydrogen, and oxygen transport membrane was fabricated from cerium gadolinium oxide and palladium (CGO/Pd) using the following method:

a) A batch of cerium gadolinium oxide powder, obtained from Rhodia, was heated in air to 1000° C. and held at that temperature for one hour. The powder was then sifted with a 60-mesh filter.

b) 6.6 g of the sifted cerium gadolinium oxide powder was mixed with 5.5 g of palladium flake, obtained from Degussa Corporation, for 30 minutes in a mortar and pestle.

c) Approximately 6 g of the mixture was loaded into a cylindrical dye (1.25 inch diameter) and compressed to 26,000 lbs. using a Carver Laboratory Press (Model #3365).

d) The CGO/Pd disc was heated in air to 1300° C. and held at that temperature for 4 hours.

The sintered membrane was placed between two gold rings and heated to 900° C. at 0.5° C./minute. The sintered membrane was sealed with gold rings into a two-zone flow reactor. While in this example a disc reactor was used, the principles of operation are the same for numerous reactor geometries including tube or cylindrical shaped reactors.

One side of the membrane was exposed to a flow air and the opposite side was exposed to a hydrocarbon mixture of steam and alkane feed. Product from the hydrocarbon side was analyzed by gas chromatography. Feeds and effluent compositions are given in Table II. Membrane reactor molar hydrogen flows and RHI were calculated using the formulas described above.

The RHI for the effluents from the membrane reactors for ethane, propane are butane feeds were 0.94, 0.96, and 0.98, respectively. These values of RHI are all significantly below 1.0, demonstrating that over a wide range of different feedstocks, processes of the invention produce membrane reactor effluents with an RHI less than 1.0.

TABLE II
Membrane Reactor Feed and Effluent Data for Example 1

| | Membrane Reactor | | | Membrane Reactor | | |
|---|---|---|---|---|---|---|
| | Feed | | | Effluent | | |
| Experiment | 157-18 CGO/ | 157-9 CGO/ | 158-5 | 157-18 | 157-9 | 158-5 |
| Material | Pd | Pd | CGO/Pd | CGO/Pd | CGO/Pd | CGO/Pd |
| Component | | | | | | |
| Flow, g/min | | | | | | |
| H2 | 0 | 0 | 0 | 0.011 | 0.002 | 0.002 |
| CO | 0 | 0 | 0 | 0.002 | 0.005 | 0.005 |
| C1 | 0 | 0 | 0 | 0.024 | 0.125 | 0.118 |
| Acetylene | 0 | 0 | 0 | 0.006 | 0.008 | 0.005 |
| Ethylene | 0 | 0 | 0 | 0.193 | 0.209 | 0.059 |
| Ethane | 0.286 | 0 | 0 | 0.034 | 0.012 | 0.008 |
| Propadiene | 0 | 0 | 0 | 0.007 | 0.013 | 0.003 |
| Propylene | 0 | 0 | 0 | 0.003 | 0.043 | 0.137 |
| Propane | 0 | 0.450 | 0 | 0 | 0.025 | 0 |
| Butadiene | 0 | 0 | 0 | 0 | 0.002 | 0.036 |
| Butene | 0 | 0 | 0 | 0 | 0 | 0.100 |
| Butane | 0 | 0 | 0.593 | 0 | 0 | 0.117 |
| Pentene | 0 | 0 | 0 | 0.005 | 0.005 | 0.004 |
| RHI | | | | 0.94 | 0.96 | 0.98 |

### Example 2 & Comparative Example C

Benefits that reactor effluents of relatively lower RHI values have for downstream processing of the effluent to recover value added products are demonstrated by the examples. In particular, the process according to the invention demonstrate that less compressor horsepower is required to compress the reactor effluent over a given pressure range, and demonstrate that a greater fraction of the desirable olefin can be recovered into a liquid condensate product at a given temperature and pressure.

This example was prepared using available thermodynamic and physical property data and a commercially available process simulation package for the relevant thermodynamic and physical property calculations.

Reactor effluent from a reactor for the dehydrogenation of propane was used to represent current practice olefin production methods that result in a relatively high-RHI reactor effluent. Pure propane feed was used and the reactor provided 60 percent conversion of the propane to propylene. In addition, a minor fraction of the propane thermally cracked to produce ethylene and methane. The composition and flow rate of the reactor effluent for this system is given as the High-RHI effluent in Table III. The reactor effluent flow rate in Table III corresponds to the production of approximately 250,000 metric tons of propylene per year. A low-RHI effluent was produced by removing 20 percent of the dihydrogen (molecular hydrogen) formed in the reactor, for example through the use of a hydrogen ion transport membrane reactor as described in Example 1. The Low-RHI effluent composition is also given in Table III. The RHI value of the effluent is also provided in Table III, assuming no dihydrogen or olefins existed in the reactor feed. The low-RHI effluent has an RHI of 0.8 while the high-RHI effluent (the one with no hydrogen removed) has an RHI of 1.0. Note that this example uses a low-RHI effluent with an RHI of 0.8 in order to provide a valid comparison case with other examples herein. In practice the RHI of a membrane reactor effluent may be higher and will depend on the hydrogen removal characteristics of the membrane material, the design of the reactor, and the heat balance requirements of the process.

The commercial process simulation package was used to determine the power required to isentropically compress the high- and low-RHI reactor effluents from the reactor outlet pressure of 50 psia to a final pressure of 250 psia. For the high-RHI effluent, a compressor power of 4140 HP was required, while for the low-RHI effluent only 3654 HP was required. This demonstrates that for a given olefin production rate, the 0.8 RHI reactor effluent saves approximately 12 percent in effluent compressor horsepower. More efficient membrane reactors (i.e. reactors which produce streams with RHIs lower than 0.8) would provide higher energy savings than shown in this example.

TABLE III
Reactor Effluent Compositions

| | Example C | Example 2 |
|---|---|---|
| Temperature, ° C. | 37.8° | 37.8° |
| Pressure, psia | 50 | 50 |
| Total Flow, mol/hr | 4070 | 3321 |

| Compound | Mole Fraction | Mole Fraction |
|---|---|---|
| Propane | 0.227 | 0.245 |
| Propylene | 0.368 | 0.397 |
| Methane | 0.018 | 0.020 |
| Dihydrogen | 0.368 | 0.318 |
| Ethylene | 0.180 | 0.020 |
| RHI Value | 1.00 | 0.80 |

The same process simulation package was then used to calculate the fraction of propylene recovered to the liquid phase after chilling the compressed reactor effluent to various final temperatures between 37.8° C. and negative 73.3° C. The results are represented in the FIGURE, which shows the fraction of the propylene in the reactor effluent that is recovered to the liquid phase after chilling to a given temperature as a function of the final temperature. In this FIGURE the high-RHI effluent is represented by open squares and the low-RHI effluent is represented by closed circles. It is clear that for a given fractional propylene recovery, a lower temperature is required for the high-RHI effluent. For example, to recover 75 percent of the propylene in the reactor effluent, the high-RHI effluent requires chilling to approximately negative 10° C. while the low-RHI effluent requires chilling to only negative 3.9° C. It is well known that less refrigeration system energy is required to chill to negative 3.9° C. than to chill to negative 10° C. Therefore in addition to the reactor effluent compressor energy savings detailed above, the low-RHI effluent also reduces the refrigeration energy that is required to recover the desired olefin product. Once again, more efficient membrane reactors (i.e. reactors which produce streams with RHIs lower than 0.8) would provide a larger shift in the condensing curve and therefore higher energy savings than shown in this example.

That which is claimed is:

1. A process for chemical conversion of volatile organic compounds to value added products, which process comprises:
(A) Providing a flow reactor comprising plurality of reaction zones each having at least one inlet for flow of fluid in contact with a solid state membrane and at least one outlet for flow of effluent from the reaction zone;
(B) Introducing a feedstream comprising volatile alkane compounds, substantially free of dihydrogen and dioxygen into all or a portion of the reaction zones;
(C) Converting, at elevated temperatures, one or more volatile compound in the feedstream to products of conversion comprising corresponding value added organic products, carbonaceous co-products, and hydrogen;
(D) Permitting at least a portion of the hydrogen co-product to be selectively conveyed out of one or more of the reaction zones through the solid membrane, thereby obtaining a gaseous effluent from the reaction zone that is characterized by a Relative Hydrogen Index value of less than 1.0; and
(E) Compressing at least a gaseous portion of the effluent from the reaction zone, cooling the compressed effluent gas to form a liquid fraction rich in organic products and a dihydrogen-rich gaseous fraction, and recovering value added hydrocarbon products from the organic-rich liquid fraction.

2. The process according to claim 1 wherein the membrane comprises at least one metal selected from the group consisting of silver, palladium, platinum, gold, rhodium, titanium, nickel, ruthenium, tungsten, and tantalum.

3. The process according to claim 1 wherein the membrane comprises a ceramic selected from the group consisting of a praseodymium-indium oxide mixture, niobium-titanium oxide mixture, titanium oxide, nickel oxide, tungsten oxide, tantalum oxide, ceria, zirconia, magnesia, or a mixture thereof.

4. The process according to claim 1 wherein the membrane comprises a multiphasic composition which in the form of a solid state membrane demonstrates an ability to selectively convey electrons, hydrogen and oxygen between different gaseous mixtures, the multiphasic composition comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity.

5. The process according to claim 4 wherein the multiphasic composition, in the form of a solid state membrane, demonstrates an ability to simultaneously convey a flux of hydrogen and, counter-current thereto, a flux of oxygen.

6. The process of claim 5 which further comprises:
Permitting a predetermined amount of oxygen, in the form of oxygen ions, to be selectively conveyed into the reaction zone through the solid state membrane.

7. A process for chemical conversion of volatile organic compounds to value added products, which process comprises:
(A) Providing a flow reactor comprising one or more reaction zone having at least one inlet for flow of fluid and at least one outlet for flow of effluent from the reaction zones;
(B) Introducing a feedstream comprising volatile alkane compounds, substantially free of dihydrogen and dioxygen into all or a portion of the reaction zones;
(C) Converting, at elevated temperatures, one or more alkane hydrocarbon in the feedstream to corresponding value added alkene hydrocarbons, carbonaceous co-products, and hydrogen; and
(D) Permitting at least a portion of the hydrogen co-product to be selectively conveyed from the reaction zone through a dense, hydrogen-permeable membrane, thereby obtaining an effluent from the reaction zone that is characterized by a Relative Hydrogen Index value of less than 1.0.

8. The process of claim 7 which further comprises; permitting a predetermined amount of oxygen, in the form of oxygen ions, to be selectively conveyed into the reaction zone through a dense, oxygen-permeable membrane.

9. The process of claim 8 which further comprises; compressing at least a gaseous portion of the effluent from the reaction zone, and cooling the compressed effluent gas to form an alkene-rich liquid fraction and a dihydrogen-rich gaseous fraction; and recovering value added alkene hydrocarbon products from the organic-rich liquid fraction.

10. The process of claim 7 which further comprises; compressing at least a gaseous portion of the reaction zone effluent to provide a compressed effluent gas at an absolute pressure greater than 1.5 times the absolute pressure of uncompressed effluent, and cooling the compressed effluent gas to form a organic-rich liquid fraction and a dihydrogen-rich gaseous fraction.

11. A process for chemical conversion of volatile organic compounds to value added products, which process comprises:
(A) Providing a flow reactor comprising one or more reaction zone having at least one inlet for flow of fluid and at least one outlet for flow of effluent from the reaction zones;
(B) Introducing a feedstream comprising volatile alkane compounds, substantially free of dihydrogen and dioxygen into all or a portion of the reaction zones;
(C) Converting, under conditions of conversion including elevated temperatures, at least 75 percent of one or more alkane hydrocarbon in the feedstream to corresponding value added alkene hydrocarbons, carbonaceous co-products and hydrogen; and
(D) Permitting at least a portion of the hydrogen co-product to be selectively conveyed from the reaction zone through a dense, hydrogen-permeable membrane, thereby obtaining an effluent from the reaction zone that is characterized by a Relative Hydrogen Index value of less than 1.0.
(F) Compressing at least a gaseous portion of the effluent from the reaction zone, cooling the compressed effluent gas to form a liquid fraction rich in organic products and a dihydrogen-rich gaseous fraction, and recovering value added hydrocarbon products from the organic-rich liquid fraction.

12. The process according to claim 11 wherein the feedstream comprises one or more volatile alkane compound having from about 1 to about 8 carbon atoms, and the conversions are carried out at elevated temperatures in a range from about 400° C. to about 900° C. and pressures in a range upward from about 15 psia to about 500 psia.

13. The process according to claim 11 wherein the recovered value added hydrocarbon products comprise at least member of the group consisting of ethylene, propylene, and isomers of butene.

14. The process according to claim 11 wherein the membrane comprises a multiphasic composition which in the form of a solid state membrane demonstrates, under conditions of conversion, an ability to selectively convey electrons, hydrogen and oxygen between different gaseous mixtures.

15. The process according to claim 14 wherein the multiphasic composition comprising two or more phases bound to one another wherein at least one of the bound phases demonstrates an ability to selectively convey hydrogen, another phase demonstrates an ability to selectively convey oxygen ions between different gaseous mixtures, and one or more of the phases demonstrates electronic conductivity.

16. The process of claim 11 wherein a flux of the co-product hydrogen conveyed through the membrane, from the first surface to the second surface of membrane is at least 1 cm³/min. at standard conditions per cm² of membrane area.

17. The process according to claim 11 wherein the feedstream comprises one or more volatile alkane compound having from about 1 to about 4 carbon atoms, and the conversions are carried out at elevated temperatures in a range from about 400° C. to about 900° C. and pressures in a range upward from about 15 psia to about 150 psia.

18. The process of claim 11 wherein the recovery of value added hydrocarbon products from the organic-rich liquid fraction provides a stream of unconverted alkane hydrocarbon and a stream of corresponding purified alkene hydrocarbon, and the process further comprises introducing all or a portion of the unconverted alkane stream into at least a portion of the reaction is zones.
